(19) United States
(12) Patent Application Publication
(10) Pub. No.: US 2025/0327024 A1
(43) Pub. Date: Oct. 23, 2025

(54) SPODOPTERA FRUGIPERDA PUPA OVARY CELL LINE WITH HIGH BACULOVIRUS PRODUCTION, AND CONSTRUCTION AND USE THEREOF

(71) Applicant: INSTITUTE OF ZOOLOGY, CHINESE ACADEMY OF SCIENCES, Beijing (CN)

(72) Inventors: Huan ZHANG, Beijing (CN); Yan TONG, Beijing (CN); Qian MENG, Beijing (CN); Qilian QIN, Beijing (CN); Xuan LI, Beijing (CN); Jihong ZHANG, Beijing (CN); Hongtuo WANG, Beijing (CN); Lin MIAO, Beijing (CN); Li GUO, Beijing (CN)

(73) Assignee: INSTITUTE OF ZOOLOGY, CHINESE ACADEMY OF SCIENCES, Beijing (CN)

(21) Appl. No.: 18/726,644

(22) PCT Filed: Jan. 4, 2023
(86) PCT No.: PCT/CN2023/070392

§ 371 Date: Jul. 3, 2024

(30) Foreign Application Priority Data
Jan. 4, 2022 (CN) 202210005201.5

(51) Int. Cl. C12N 5/07; A01N 63/40; A01P 7/04; C12N 7/00; C12N 15/86

(52) U.S. Cl. CPC C12N 5/0601; A01N 63/40; A01P 7/04; C12N 7/00; C12N 15/86; C12N 2710/14131; C12N 2710/14143; C12N 2710/14152

(57) ABSTRACT

Provided is a Spodoptera frugiperda pupa ovary cell line with high Baculovirus production. The cell line has the name of IOZCAS-Sf-1 and the deposit number of CGMCC No. 21014. Further disclosed are a method for preparing the cell line and the use of the cell line in the large-scale growth of the Baculovirus. The cell line can be used for replicating the Baculovirus, for the large-scale production of Baculovirus insecticides and for constructing a Baculovirus expression vector system.

### REFERENCE TO AN ELECTRONIC SEQUENCE LISTING

A sequence listing in electronic (XML file) format was filed with this application and incorporated herein by reference. The name of the XML file is “Sequence-Listing-0919”; the file was created on Jan. 30, 2025; the size of the file is 2,689 bytes.

### TECHNICAL FIELD

The application belongs to the field of biotechnology. The present application relates to an insect cell line, particularly a cell line derived from insect ovary tissue and highly sensitive to baculovirus. The present application further relates to the method of constructing the cell line, and the use of the cell line on the large-scale growth of baculovirus.

### BACKGROUND ART

According to reports, since the first insect cell line was successfully established in 1965, more than 700 insect cell lines have been established in the past 60 years. They come from more than 170 kinds of insects including Lepidoptera, Diptera, Homoptera, Hymenoptera, Orthoptera and Coleoptera, with the majority coming from Lepidoptera and Diptera. Insect cell lines, as research materials, have always been an important tool for scientific research in physiology, developmental biology, cell biology, molecular biology and biochemistry, and insect cells, as an important part of the baculovirus expression vector system, express a large number of exogenous proteins of great economic or scientific significance. At the same time, as a bioreactor to amplify insect baculoviruses for use as biopesticides, especially to amplify recombinant baculovirus insecticides containing exogenous genes, insect cells also play an important role.

A large number of commercial cell lines derived from Lepidoptera insects have been widely used, for example, IPLB-Sf21-AE (Sf-21) derived from Spodoptera frugiperda and its clone Sf-9, and Tn-368 derived from Trichoplusia ni and its clone Tn-5B1-4 (trade name: High Five) have been successfully applied to the expression and production of recombinant proteins.

So far, there are about 24 originally established cell lines from Spodoptera frugiperda recorded in the Cellosaurus database (https://web.expasy.org/cellosaurus), in which the commercialized Sf-21 and its clone Sf-9 has been widely used in the expression and production of recombinant proteins due to its high virus yield and fast proliferation. However, in the process of protein expression, there are still problems such as low expression of some types of proteins. Cell lines derived from different species of insects or from different tissue sites of the same insect species vary in their ability to amplify viruses or express recombinant proteins. Therefore, establishing and screening new and more baculovirus-sensitive cell lines (strains) is a very necessary work with theoretical and practical significance. At present, there is still a great demand for cell lines from insects in various biological experiments, and it is of great significance to use them to construct a more efficient baculovirus expression vector system.

### SUMMARY

Aiming at the problem that it is still necessary to further develop insect cell lines sensitive to baculovirus, the present application provides a Spodoptera frugiperda pupa ovary cell line with high baculovirus production derived from Spodoptera frugiperda. The application also provides a use of a Spodoptera frugiperda pupa ovary cell line with high baculovirus production in large-scale growth of baculovirus.

Specifically, in an aspect, the present application relates to a Spodoptera frugiperda pupa ovary cell line with high baculovirus production, wherein the cell line has a name of IOZCAS-Sf-1, and a deposit number of CGMCC No. 21014.

In an aspect, the present application relates to a method for preparing the Spodoptera frugiperda pupa ovary cell line with high baculovirus production, comprising the following steps:
1) cultivating Spodoptera frugiperda ovary cells with a cell culture fluid; and
2) obtaining a Spodoptera frugiperda ovary cell line with high baculovirus production.

In some embodiments, the cell culture medium fluid is a mixture of an insect cell culture fluid and an antibiotic. The antibiotic is penicillin and streptomycin. The cell culture fluid is further supplemented with fetal bovine serum. A content of the penicillin is 100 U/mL; a content of the streptomycin is 100 g/mL. A content of the fetal bovine serum is 10% by volume of the cell culture fluid. The cell culture fluid has a pH of 5.8-6.8. The insect cell culture fluid is selected from: Insect-XPRESS™, TNM-FH, Grace's, Sf-900, TC-100, IPL-41 or Ex-Cell 400.

In an aspect, the present application relates to use of the Spodoptera frugiperda pupa ovary cell line with high baculovirus production in the production of baculovirus.

In some embodiments, the baculovirus is Autographa californica nuclear polyhedrosis virus (AcMNPV).

In an aspect, the present application relates to use of the Spodoptera frugiperda pupa ovary cell line with high baculovirus production in the preparation of a baculovirus insecticide.

In an aspect, the present application relates to use of the Spodoptera frugiperda pupa ovary cell line with high baculovirus production in the preparation of a baculovirus expression vector system.

The Spodoptera frugiperda pupa ovary cell line with high baculovirus production provided by the present application can be used to replicate baculovirus for large-scale growth of baculovirus and large-scale production of baculovirus insecticide. This cell line can also be applied to the construction of a baculovirus expression vector system, which can express recombinant proteins with high commercial and scientific value.

### BRIEF DESCRIPTION OF THE DRAWINGS

The embodiments of the present application were described in details in conjunction with the accompanying drawings below, wherein:

FIG. 1 shows the adherent culture of the ovary tissue of the Spodoptera frugiperda during the process of the primary culture and it can be seen that the cells are free from the tissue, indicating that the cells are derived from the ovary tissue of the Spodoptera frugiperda pupa, with a scale of 100 μm;

FIG. 2 shows the cell morphology of the Spodoptera frugiperda pupa ovary cell line IOZCAS-Sf-1 of the present application, with a scale of 50 μm;

FIG. 3 shows the alignment results of the COI gene sequence of the Spodoptera frugiperda pupa ovary cell line IOZCAS-Sf-1 of the present application with the sequence of COI gene of the Spodoptera frugiperda larval blood cells, commercialized Sf9 cell line on NCBI;

FIG. 4 shows the growth curve of the Spodoptera frugiperda pupa ovary cell line IOZCAS-Sf-1 of the present application;

FIG. 5 shows the flow cytometry detection of chromosome ploidy of the Spodoptera frugiperda pupa ovary cell line IOZCAS-Sf-1 of the present application;

FIG. 6 shows a large number of virus polyhedrons were obtained after IOZCAS-Sf-1 was infected with Autographa californica nuclear polyhedrosis virus (AcMNPV), with a scale of 100 μm.

### Preservation of Biological Material

The Spodoptera frugiperda pupa ovary cell line with high Baculovirus production IOZCAS-Sf-1, classified as Spodoptera frugiperda pupa ovary cell line, has been deposited on Nov. 25, 2020 in the China General Microbiological Culture Collection Center (CGMCC), the address of which is: No. 3, Yard 1, Beichen West Road, Chaoyang District, Beijing, P.R. China, 100101, with a deposit number of CGMCC No. 21014.

### DETAILED DESCRIPTION

The present application will be described in detail in conjunction with the accompanying drawings and specific examples. It should be understood that the following examples are only used to illustrate the present application and are not intended to limit the scope of the present application.

In an aspect, the present application provides a Spodoptera frugiperda pupa ovary cell line with high baculovirus production, wherein the cell line has a name of IOZCAS-Sf-1, and a deposit number of CGMCC No. 21014, it is classified as Spodoptera frugiperda pupa ovary cell line, and was deposited in the China General Microbiological Culture Collection Center on Nov. 25, 2020.

In another aspect, the present application provides a method for preparing the Spodoptera frugiperda pupa ovary cell line with high Baculovirus production, comprising steps of: 1) cultivating Spodoptera frugiperda ovary cells with a cell culture fluid; and 2) obtaining a Spodoptera frugiperda ovary cell line with high baculovirus production.

In a specific embodiment, a method for preparing the Spodoptera frugiperda pupa ovary cell line with high Baculovirus production comprises steps of: (1) immersing the female Spodoptera frugiperda pupa in ethanol solution for 10-20 minutes to carry out surface disinfection, then washing the insect with sterile distilled water, and then blotting the surface of the insect body; (2) dissecting the insect, and taking out the intact Spodoptera frugiperda pupa ovary tissue; (3) washing the tissue obtained in (2) with physiological saline for 2˜3 times and then with cell culture fluid, placing the tissue in a cell culture bottle rinsed with 1 mL of cell culture fluid after washing, capping the bottle, and placing in a cell incubator at 27° C. for 24 hours of culture; (4) adding an appropriate amount of cell culture fluid, so that the tissue piece is completely or mostly immersed in the culture fluid, and culturing under the same conditions as step (3) for about 21 days to obtain a large number of primary cells; (5) feeding nitrogen into the three-gas incubator to replace oxygen, and detecting the oxygen concentration in the incubator by an oxygen sensor, to control and reduce the oxygen content in the incubator to 5%; meanwhile, replacing the airtight bottle cap of the cell culture bottle with a ventilating bottle cap to allow the gas inside the cell culture bottle to circulate with the gas in the incubator, achieving the effect of hypoxia, and carrying out hypoxia culture on the primary cells of the insect at 27° C. for 9 days; (6) replacing the cell culture fluid and the cap of the culture bottle, and carrying out normoxia culture at 27° C. for 10 to 30 days, such as 10 days, 11 days, 12 days, 13 days, 14 days, 15 days, 20 days, 25 days or 30 days; (7) cultivating sequentially the reoxygenated cells, allowing the living cells to continuously proliferate until they fill the bottom of the cell culture bottle and begin passaging, thereby successfully establishing a Spodoptera frugiperda cell line, to obtain the Spodoptera frugiperda pupa ovary cell line with high baculovirus production of the present application; in which the whole process is performed under sterile conditions.

In a specific embodiment, the cell culture fluid used in the above-mentioned method of the present application is the mixture of conventional insect cell culture fluid and penicillin, streptomycin and fetal bovine serum, and the pH of the cell culture fluid is 5.8 to 6.8, for example, 5.8 to 5.9, 5.9 to 6.0, 6.0 to 6.1, 6.0 to 6.2, 6.0 to 6.3, 6.0 to 6.4, 6.0 to 6.5, 6.0 to 6.6, 6.0 to 6.7, 6.0 to 6.8. The insect cell culture fluid is commercial insect cell culture fluid Insect-XPRESS™, TNM-FH, Grace's, Sf-900, TC-100, IPL-41, or Ex-Cell 400. Usually, the content of penicillin in the cell culture fluid is 10 U/mL to 500 U/mL, preferably 100 U/mL; the content of streptomycin is 10 g/mL to 500 g/mL, preferably 100 g/mL; the content of animal serum is the 5%-20% by volume of the cell culture fluid, preferably 10% by volume of the cell culture fluid.

In another aspect, the present application provides a use of the Spodoptera frugiperda pupa ovary cell line with high baculovirus production in the production of baculovirus.

In a specific embodiment, the baculovirus is Autographa californica nuclear polyhedrosis virus (AcMNPV), Spodoptera frugiperda nuclear polyhedrosis virus (SfMNPV).

In another aspect, the present application provides use of the Spodoptera frugiperda pupa ovary cell line with high baculovirus production in the preparation of a baculovirus insecticide.

In another aspect, the present application provides use of the Spodoptera frugiperda pupa ovary cell line with high baculovirus production in the preparation of a baculovirus expression vector system.

### EXAMPLE

### Example 1. Establishment of Spodoptera frugiperda Pupa Ovary Cell Line

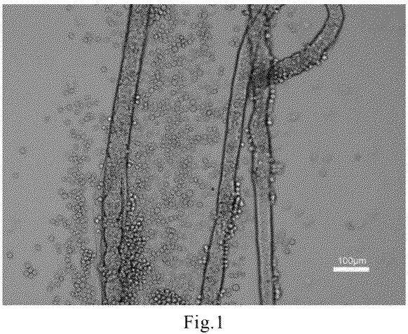

The mid-developmental Spodoptera frugiperda pupa (raised by the Entomology Virology Research Group of the State Key Laboratory of Comprehensive Management of Agricultural Pests and Rodents, Institute of Zoology, Chinese Academy of Sciences) is immersed in 75% ethanol solution for 10 minutes to carry out surface disinfection. The insect was dissected, and the ovary tissue was taken out, which was kept as intact as possible during the manipulation. The tissue was washed with physiological saline for 2˜3 times and with cell culture fluid (Insect-XPRESS™ (BioWhittake, Lonza, Walkersville, MD, USA, 12-730Q), containing 100 U/mL penicillin, 100 g/mL streptomycin and 10% (v/v) fetal bovine serum (BI, Beit Haemek, Israel, 04-001-1), pH=6.2) for 1˜2 times, and was placed in a 25 cm² cell culture bottle rinsed with 1 mL of the above-mentioned cell culture fluid, which was placed in a cell incubator at 27° C. without light for 24 hours of culture. Then the tissue was added with 3 mL of the above-mentioned cell culture fluid and cultured under the same conditions. After the first 2-3 days of this operation, it can be observed that a large amount of individual cells were free around the tissue, and gradually expand to the periphery (FIG. 1). Cells proliferated gradually to fill the whole culture bottle in about 21 days of primary culture, and the airtight bottle cap is replaced with a ventilating bottle cap. The culture bottle was put into a three-gas incubator, and introduced nitrogen to control the oxygen concentration to 5%. After 9 days of culture under this hypoxia condition, the fresh culture fluid and the airtight bottle cap were replaced. After about 30 days of culture under reoxygenated conditions, newly proliferated cells along with the whole cell culture fluid were sucked out and placed into the new culture bottle, into which was added 2 mL of the new cell culture fluid mentioned above. The cell line was initially successfully established. The second bottle passage began on the 7th day after the initiation of cell passage, and then the passage time was gradually shortened. By the 8th passage, cell growth began to stabilize, and finally the cell line was named IOZCAS-Sf-1. IOZCAS-Sf-1 was deposited on Nov. 25, 2020 in the China General Microbiological Culture Collection Center, with a deposit number of CGMCC No. 21014.

### Example 2. Observation and Determination of Biological Characteristics of IOZCAS-Sf-1

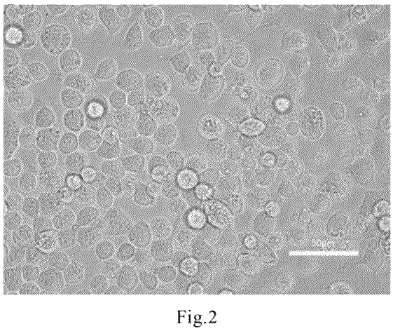

(1) Morphological features: Upon microscopic observation, the IOZCAS-Sf-1 cell line includes cells suspended in the culture fluid and adherent cells, with two types of cell shapes: circular and spindle. In IOZCAS-Sf-1, circular cells account for 69.2%, with an average diameter of 7.98±0.105 (mean±standard deviation) μm; and spindle cells account for 30.8%, with a length of 10.13±0.127 μm and a width of 6.50±0.0.101 μM (FIG. 2).

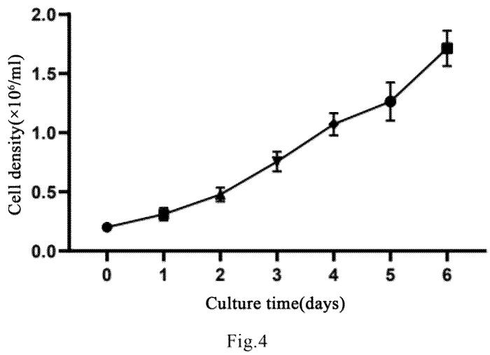

(2) Growth of cells: IOZCAS-Sf-1 was inoculated into a 24-well plate at a concentration of 2× 10⁵ cells/mL, and cultured at 27° C. The growth curve and population doubling time of the 20th generation of cells were determined according to the method in McIntosh and Ignoro, 1989 (McIntosh, A. H. and C. M. Ignoffo J. Invertebr. Pathol. 1989, 54: 97˜102). That is, the cell concentration was determined every 24 hours, a growth curve was plotted (FIG. 4), and the cell population doubling time was calculated according to the formula T-tlg2/[lg(N/N₀)] (wherein T=the time required for the average doubling in the logarithmic phase; t=the time from inoculation to the determination of the cell number; N₀=the cell number at the time of inoculation; N=the total cell number measured at time t). The 20th generation of IOZCAS-Sf-1 cell line showed a cell population doubling time of 46.47 hours in Insect-XRESS™ culture fluid containing 10% fetal bovine serum, 100 U/mL penicillin, and 100 g/mL streptomycin. At present, the cell line has been passaged for more than 30 generations.

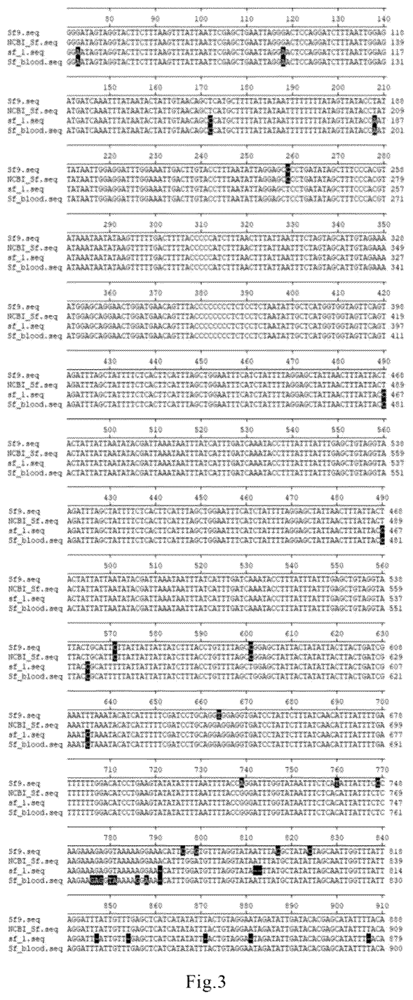

(3) Identification by COI sequencing method: The cell line IOZCAS-Sf-1 was indeed derived from the Spodoptera frugiperda, without contamination from other cell lines and Sf9 cells. The DNA extracted from IOZCAS-Sf-1 was amplified by PCR with the blood DNA of Spodoptera frugiperda larvae, with the COI gene amplification primers used being (upstream primer: 5′-3′ SEQ ID NO 1: ID NO 2: TTCGAGCTGAATTAGGACTC, downstream primer: 5′-3′ SEQ GATGTAAATATATATGCTGT) (FIG. 3).

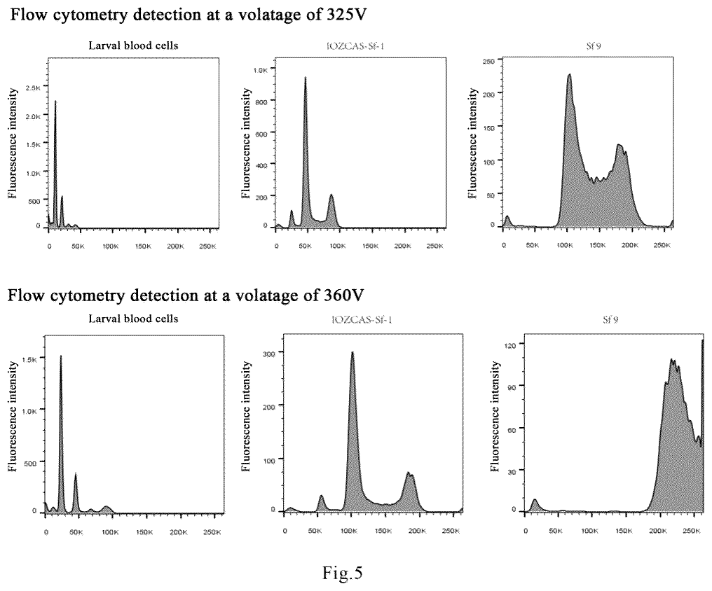

(4) Detection of cell chromosome multiples by flow cytometry. The Spodoptera frugiperda larval blood cells were used as diploid control. Cells were collected and fixed overnight at 4° C. in 70% ethanol, and incubated at 37° C. for 30 min with 0.1 mg/mL propidium iodide (PI) (Solarbio, Beijing, CA1630-500T). After filteration, detection was performed by flow cytometry (BD Biosciences, East Rutherford, NJ, USA, LSR Fortessa). Under the same voltage, with larval blood cells as diploid control, the first fluorescence intensity peak of IOZCAS-Sf-1 is 4 times that of larval blood cells, while Sf9 cells were polyploid control. Therefore, it was determined that IOZCAS-Sf-1 was an octoploid (FIG. 5).

(5) Cryopreservation and resuscitation: The cells were cryopreserved by the traditional method of cell cryopreservation, to preserve the genetic resources thereof, which can be successfully resuscitated.

### Example 3. Determination of Sensitivity of IOZCAS-Sf-1 to Baculovirus

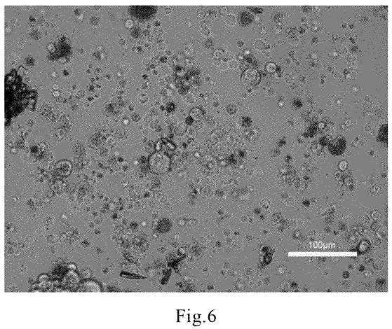

IOZCAS-Sf-1 was sensitive to Autographa californica nuclear polyhedrosis virus (AcMNPV): the AcMNPV budding virus particle BV (the Spodoptera frugiperda larvae fed on AcMNPV polyhedron for 3 days, the hemolymph of the larvae was taken, and the supernatant solution after removing the blood cells was preserved by the Institute of Zoology, Chinese Academy of Sciences) was inoculated with IOZCAS-Sf-1 at a concentration of MOI (multiplicity of infection)=1. After 7 days of culture, cells and virus polyhedrons can be harvested, and the infection rate is about 56.4%. Under an inverted phase contrast microscope, typical cellular pathological features can be observed, that is, the cell nuclei was enlarged and contained a large number of viral polyhedron particles (FIG. 6).

What is claimed is:

1. A Spodoptera frugiperda pupa ovary cell line with high baculovirus production, wherein the cell line has a name of IOZCAS-Sf-1, and a deposit number of CGMCC No. 21014.

2. A method for preparing the ovary cell line according to claim 1, comprising steps of:
1) cultivating Spodoptera frugiperda ovary cells with a cell culture fluid; and
2) obtaining a Spodoptera frugiperda ovary cell line with high baculovirus production.

3. The method according to claim 2, wherein the cell culture fluid is a mixture of an insect cell culture fluid and an antibiotic.

4. The method according to claim 3, wherein the antibiotic is penicillin and streptomycin.

5. The method according to claim 2, wherein the cell culture fluid is further supplemented with fetal bovine serum.

6. The method according to claim 4, wherein a content of the penicillin is 10 U/mL to 500 U/mL, preferably 100 U/mL; a content of the streptomycin is 10 g/mL to 500 g/mL, preferably 100 g/mL.

7. The method according to claim 5, wherein a content of the fetal bovine serum is 5%-20% by volume of the cell culture fluid, preferably 10% by volume of the cell culture fluid.

8. The method according to claim 2, wherein the cell culture fluid has a pH of 5.8-6.8.

9. The method according to claim 2, wherein the insect cell culture fluid is selected from: Insect-XPRESS™, TNM-FH, Grace's, Sf-900, TC-100, IPL-41 or Ex-Cell 400.

10. Use of the ovary cell line of claim 1 in the production of baculovirus.

11. The use according to claim 10, wherein the baculovirus is Autographa californica nuclear polyhedrosis virus (AcMNPV) and Spodoptera frugiperda nuclear polyhedrosis virus (SfMNPV).

12. Use of the ovary cell line of claim 1 in the preparation of a baculovirus insecticide.

13. Use of the ovary cell line of claim 1 in the preparation of a baculovirus expression vector system.


SEQUENCE LISTING (2 sequences)

SEQ ID NO: 1 - DNA, 21 bp, synthetic construct
  source 1..21: mol_type = other DNA
ttcgagctgaattagggactc

SEQ ID NO: 2 - DNA, 20 bp, synthetic construct
  source 1..20: mol_type = other DNA
gatgtaaaatatgctcgtgt